(19) United States
(12) Patent Application Publication
(10) Pub. No.: US 2018/0056015 A1
(43) Pub. Date: Mar. 1, 2018

(54) DUAL VAPORIZER

(71) Applicant: Max Shwadchuck, Garibaldi Highlands (CA)

(72) Inventor: Max Shwadchuck, Garibaldi Highlands (CA)

(21) Appl. No.: 15/688,205

(22) Filed: Aug. 28, 2017

Related U.S. Application Data
(60) Provisional application No. 62/381,487, filed on Aug. 30, 2016.

(51) Int. Cl. A61M 15/06; A24F 47/00; A61M 15/00

(52) U.S. Cl. CPC A61M 15/06; A24F 47/008; A61M 15/0021; A61M 15/0086; A61M 2205/33; A61M 2205/123; A61M 2205/125; A61M 2205/127; A61M 2205/076

(57) ABSTRACT

A dual vaporizer is provided. The vaporizer has: a vaporizing assembly including a first chamber with a plant matter compartment for holding dry plant matter and a first heating element; a vapor cooling chamber in fluid communication with the compartment; a second chamber comprising a second heating element, the second chamber in fluid communication with the vapor cooling chamber; an e-juice chamber; a wick in fluid communication with the second chamber and the e-juice chamber, the wick coupled to the second heating element; a mouthpiece in fluid communication with the second chamber; and a power source for providing electrical power to the first heating element and the second heating element, whereby suction at the mouthpiece draws air and any vapor from the vapor cooling chamber through the second chamber.

### TECHNICAL FIELD

This invention relates to vaporizers.

### BACKGROUND

Vaporizers are devices that allow a user to inhale a substance in the form of vapor. Vaporizers may be used for therapeutic and recreational purposes.

Some vaporizers combust plant extract-containing oils or dry plant matter. Combustion forms desirable large clouds but is similar to smoking and has adverse health effects on the user's lungs.

Other vaporizers vaporize a liquid such as “e-juice” or specialized liquid plant extracts. E-juice typically contains water, nicotine, flavouring agents, and a carrier comprising propylene glycol (“PG”) and/or vegetable glycerin (“VG”). Vaporization of the carrier forms desirable large clouds, nicotine levels can be adjusted, and a wide variety of desirable flavourings are available. A drawback is that dry plant matter cannot be used.

Still other vaporizers do not combust dry plant matter but rather vaporize volatiles within the dry plant matter at temperatures below its kindling point. Little or no clouds are formed, but the lower heat used has a lower impact on the user's lungs. Heating certain dry plant matter in this manner can generate undesirable odor and/or taste.

Improved vaporizers that address one or more of the above-described deficiencies are desirable.

### BRIEF DESCRIPTION OF DRAWINGS

The accompanying drawings illustrate non-limiting example embodiments of the invention.

FIG. 1 is a schematic diagram of a dual vaporizer according to an embodiment of the invention.

FIG. 2 is a schematic diagram of a dual vaporizer according to another embodiment of the invention.

FIG. 3 is a schematic diagram of a dual vaporizer according to another embodiment of the invention.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

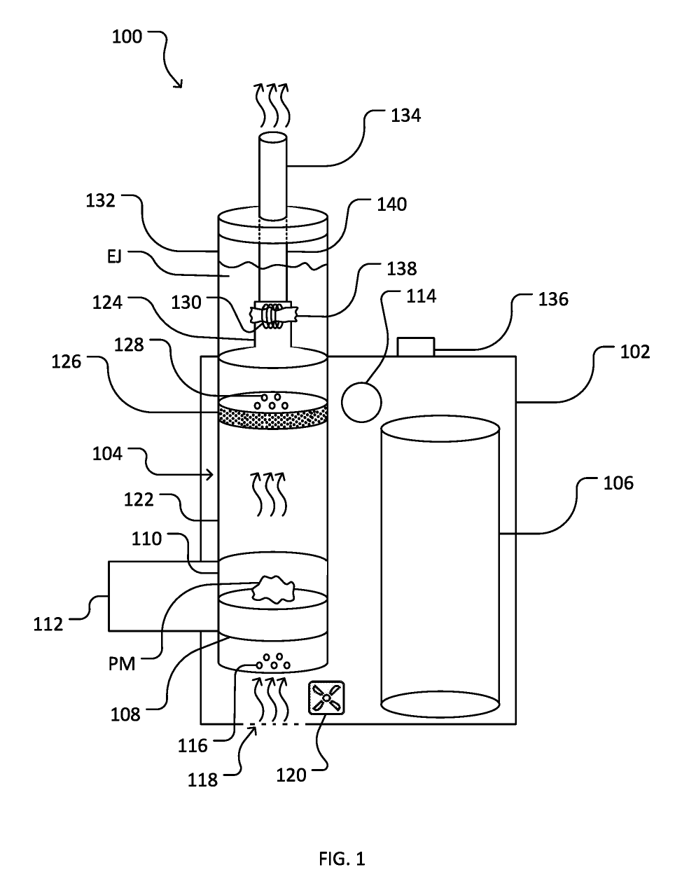

FIG. 1 shows a dual vaporizer 100 according to one embodiment of the invention. Dual vaporizer 100 has a housing 102 that partially houses a vaporizing assembly 104. In some embodiments, housing 102 may entirely house a vaporizing assembly 104 (except for mouthpiece 134). Also disposed in housing 102 is a power source 106. Power source 106 may be one or more rechargeable batteries, such as lithium ion batteries or nickel-metal hydride batteries. Power source 106 provides electrical power to various components of dual vaporizer 100 as described below.

Vaporizing assembly 104 includes components for vaporizing volatile compounds in dried plant matter PM, and components for vaporizing wet matter, namely e-juice or e-liquid EJ, wherein these components are arranged so that the output air/vapor of the dry matter vaporizer is used as the input for the wet matter vaporizer to thoroughly combine vapors from the dry matter and wet matter before inhalation at the mouthpiece. This inventive configuration provides advantages including (i) allowing desirable aroma/taste of the wet matter vapor to mask any undesirable odor/taste of the dry matter vapor and (ii) generating desirable smoke with the wet matter vapor where the dry matter vapor is smokeless.

In the following description of vaporizing assembly 104, components for vaporizing dry matter (i.e., plant matter PM volatiles) will be described first, followed by the components for vaporizing wet matter (i.e., e-juice EJ).

Vaporizing assembly 104 has a plant matter compartment 110 for holding plant matter PM. Plant matter compartment 110 is accessed by a user through access means 112. Access means 112 may be a door (as illustrated in FIG. 1), a closeable slot, or the like. Plant matter compartment 110 includes a first heating element 108 for heating and vaporizing plant matter PM volatiles. In some embodiments, plant matter PM may be provided in a specially pressed form or package insertable in plant matter compartment 110.

The plant matter PM may be marijuana-based material containing bioactive components that can be vaporized for inhalation. In some embodiments, the marijuana-based material may be used for medical purposes. In some embodiments, the marijuana-based material may be used for recreational purposes. The marijuana-based material may be derived from a cannabis plant selected from Cannabis sativa, Cannabis indica, a genetic cross between them, a self-cross or a hybrid thereof. In some embodiments, plant matter PM is free of tobacco.

First heating element 108 may be one or more heating elements in direct contact with plant matter PM. In other embodiments, first heating element 108 may not directly contact plant matter PM and instead heat plant matter PM indirectly by conduction or convection using suitable means known to those skilled in the art.

Electrical power for first heating element 108 is provided by power source 106. First heating element 108 is configured to provide sufficient heat to vaporize plant matter PM volatiles but not reach or exceed the kindling point of plant matter PM. For example, the primary bioactive components in marijuana have the following boiling points: tetrahydrocannabinol (THC): 157° C.; cannabidiol (CBD): 160-180° C.; and cannabinol (CBN): 185° C. In some embodiments, first heating element 108 therefore may be configured to directly or indirectly heat plant matter PM in plant matter compartment 110 up to 200° C., or 220° C., or 240° C., or 260° C., or 280° C.

The amount of heat released by first heating element 108 may be controlled by first heating element control unit 114. First heating element control unit 114 may be a button, a dial, a touch screen, a switch, pressure sensor, or the like. If control unit 114 is a dial, then the dial may for example have indicator marks such as “off”, “low”, “medium” and “high”, or numerical indicators, to correspond with the relative temperature settings of first heating element 108. If control unit 114 is a pressure sensor, then first heating element 108 may be activated by a user inhaling from mouthpiece 134 to create negative pressure in vaporizing assembly 104.

Heating element 108 is cooled by air from the interior of housing 102 entering vaporizing assembly 104 through vent 116, and surrounding vaporizing assembly 104. Ambient air may be drawn from outside dual vaporizer 100 into housing 102 through air intake 118. In some embodiments a cooling means 120, such as a fan or air pump, powered by power source 106, may be provided to assist in drawing ambient air into housing 102 through air intake 118 and into vaporizing assembly 104 through vent 116. In some embodiments cooling means 120 may be configured to operate whenever heating element 108 is turned on. In some embodiments, cooling means 120 may be in communication with a sensing/actuating means (not shown) that automatically turns cooling means 120 on whenever the temperature of a heating element 108 (or a region nearby heating element 108) exceeds a predetermined threshold temperature.

Vaporizing assembly 104 includes a vapor cooling chamber 122 directly above and integral with plant matter compartment 110. In some embodiments vapor cooling chamber 122 may be a separate chamber but still in fluid communication with plant matter compartment 110. Vapor cooling chamber 122 is elongated and sized to allow plant matter PM volatiles to at cool to at least a temperature that, when consumed at mouthpiece 134, does not cause discomfort to a user.

A filter or screen 126 prevents plant matter PM particles from vapor cooling chamber 122 entering coil chamber 124. Plant matter PM volatiles vapor and air from vapor cooling chamber 122 can enter coil chamber 124 through one or more holes 128 in the bottom surface of coil chamber 124 abutting filter or screen 126. In some embodiments, the bottom surface of coil chamber 124 may be formed as a filter or screen 126, obviating the need for holes 128.

Coil chamber 124 houses a second heating element 130. Second heating element 130 may be a coil, such as single coil (as shown in FIG. 1) or dual coil. Coil 130 is activated by a trigger 136. Trigger 136 may be an input such as a button, a dial, a touch screen, a switch, pressure sensor, or the like.

Coil 130 is connected to a wick 138 that extends from coil chamber 124 into e-juice chamber 132, thereby wicking e-juice EJ from e-juice chamber 132 onto coil 130 in coil chamber 124 and vaporizing the e-juice EJ. The plant matter PM volatiles vapor and air from vapor cooling chamber 122 serves as the input air for the e-juice EJ vaporization that occurs in coil chamber 124.

E-juice EJ may be any e-juice or e-liquid known to those skilled in the art. In some embodiments, e-juice EJ comprises water, flavoring agents, and a carrier that generates smoke such as comprising propylene glycol (“PG”) and/or vegetable glycerin (“VG”), and is free of nicotine.

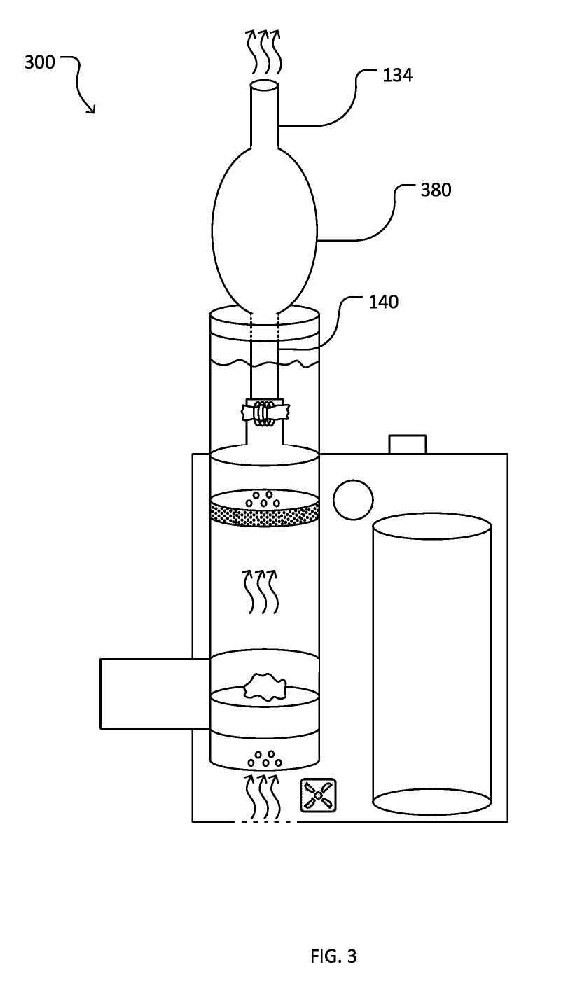

Combined vapor delivery tube 140 connects coil chamber 124 to mouthpiece 134. In some embodiments, as shown in dual vaporizer 300 in FIG. 3, a second vapor cooling chamber 380 may be provided between combined vapor delivery tube 140 and mouthpiece 134 to provide additional cooling and mixing of the vapors for user comfort and enjoyment.

In operation of a vaporizer such as dual vaporizer 100 according to an embodiment, a user inserts plant matter PM into plant matter compartment 110 and fills e-juice chamber 132 with e-juice EJ; this initial step is obviously not required if dual vaporizer 100 is pre-loaded with a sufficient amount of plant matter PM and sufficient amount of e-juice EJ intended to be consumed by the user. Next, the user heats plant matter PM by turning on first heating element 108 by activating first heating element control unit 114. As the plant matter PM is heated and reaches the boiling point of volatile compounds in the plant matter PM, the vapor of the volatiles rises up into vapor cooling chamber 122 and cools. Next the user activates trigger 136 to allow coil 130 to heat to a temperature that vaporizes e-juice EJ from e-juice chamber 132 wicked onto coil 130 by wick 138. The user then inhales at mouthpiece 134 to draw plant matter PM volatiles vapor through screen/filter 126, combine with e-juice EJ vapor generated in coil chamber 124, through combined vapor delivery tube 140, and out mouthpiece 134. In some embodiments, the foregoing method is used with medical marijuana or recreational marijuana as the plant matter PM (without any tobacco) and e-juice EJ with flavouring desired by the user (with or without any nicotine).

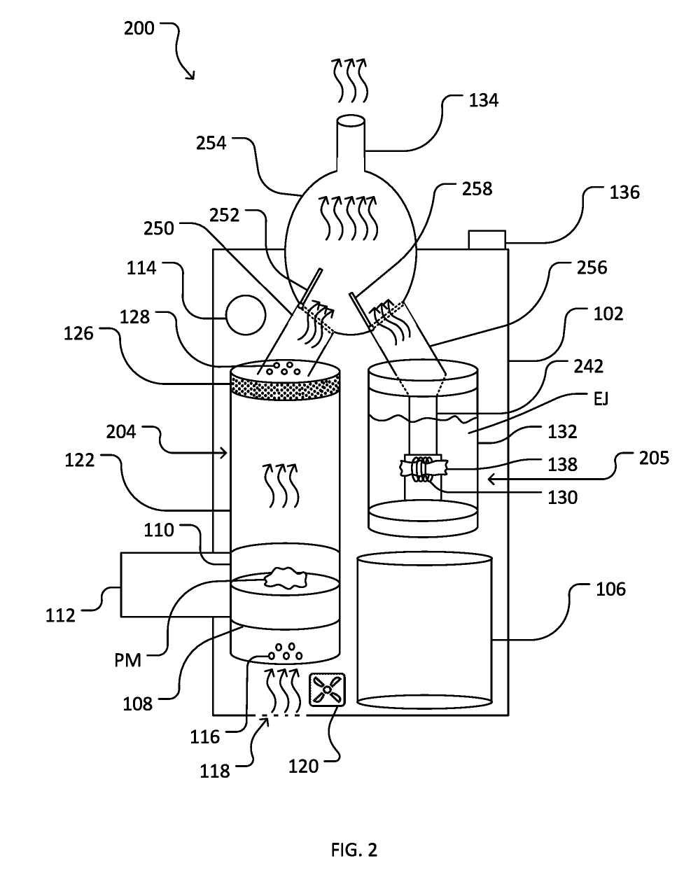

FIG. 2 shows a dual vaporizer 200 according to another embodiment. Like numbered components between dual vaporizer 100 and dual vaporizer 200 share similar functions. Unlike dual vaporizer 100, dual vaporizer 200 allows a user to regulate entry of plant matter PM volatiles vapors and e-juice EJ vapors into a combined vapor chamber 254. In particular, a connector 250 connects the downstream end of the plant matter PM volatiles vapor producing components of dual vaporizer 200, such as screen/filter 126 and holes 128, to a combined vapor chamber 254. A connector 256 connects the downstream end of the e-juice EJ vapor producing components of dual vaporizer 200, such an extended portion 242 of the coil chamber 124, also to combined vapor chamber 254. While dual vaporizer 100 achieved significant mixing of vapors by utilizing the dry matter vapor (and air) as the input for vaporization of the wet matter, dual vaporizer 200 allows for significant mixing of vapors at combined vapor chamber 254. In some embodiments, the capacity of combined vapor chamber 254 may be at least 10 mL, or 20 mL, or 30 mL, or 40 mL or 50 mL.

Valve 252 regulates entry of plant matter PM volatiles vapor into combined vapor chamber 254. Valve 258 regulates entry of e-juice vapor into combined vapor chamber 254. Valves 252, 258 may be any type of valve suitable for regulating entry of vapor into combined vapor chamber 254, such as flap valves, gate valves, butterfly valves, iris valves and the like.

In some embodiments, the valves may be configured to open upon triggering of first heating element 108 and second heating element 130. In some embodiments, the valves may be configured to allow the user to control the proportion of vapor from each source that enters combined vapor chamber 254; for example, if the user desires to consumer a higher proportion of e-juice EJ vapor over plant matter PM volatiles vapor, then a separate control unit (not shown) may be provided to allow for valve 258 to open to a greater extent than valve 252.

In some embodiments, dual vaporizers 100, 200 may be configured as personal vaporizer (i.e. an electronic cigarette or handheld vaporizer), while in other embodiments dual vaporizers 100, 200 may be configured as a desktop vaporizer.

The present invention has particular advantages in the context of vaporizing plant matter that produce undesirable odor and taste profiles when vaporized and/or do not produce significant smoke. For example, the inventor has determined that vaporization of plant matter from Cannabis plants produces an undesirable odor and does not produce much smoke. The present invention masks the odor and taste profile of vaporized Cannabis plant volatiles with the desirable aroma and flavourings of e-juice vapor by significant mixing of the vapors as described herein, thereby combining the bioactivity of Cannabis plant volatiles with the odor and taste profile of a user's preferred e-juice vapor. Further, by utilizing e-juice with carriers that produce significant smoke (e.g. comprising propylene glycol (“PG”) and/or vegetable glycerin (“VG”)), the desirable effect of significant smoke is achieved as well.

Where a component is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

This application is intended to cover any variations, uses, or adaptations of the invention using its general principles. Further, this application is intended to cover such departures from the present disclosure as come within known or customary practice in the art to which this invention pertains and which fall within the limits of the appended claims. Accordingly, the scope of the claims should not be limited by the preferred embodiments set forth in the description, but should be given the broadest interpretation consistent with the description as a whole.

1. A dual vaporizer comprising:
a vaporizing assembly comprising:
a first chamber comprising:
a plant matter compartment for holding dry plant matter; and
a first heating element;
a vapor cooling chamber in fluid communication with the compartment;
a second chamber comprising a second heating element, the second chamber in fluid communication with the vapor cooling chamber;
an e-juice chamber;
a wick in fluid communication with the second chamber and the e-juice chamber, the wick coupled to the second heating element;
a mouthpiece in fluid communication with the second chamber; and
a power source for providing electrical power to the first heating element and the second heating element,
whereby suction at the mouthpiece draws air and any vapor from the vapor cooling chamber through the second chamber.

2. A dual vaporizer according to claim 1 comprising a filter between the vapor cooling chamber and the second chamber.

3. A dual vaporizer according to claim 2 wherein the second heating element comprises one or more coils.

4. A dual vaporizer according to claim 3 comprising a second vapor cooling chamber between the second chamber and the mouthpiece.

5. A dual vaporizer comprising:
a plant matter volatile vaporizing assembly comprising:
a plant matter compartment comprising a first heating element;
a vapor cooling chamber in fluid communication with the plant matter compartment;
an e-juice vaporizing assembly comprising:
a coil chamber comprising a second heating element;
an e-juice chamber;
a wick in fluid communication with the coil chamber and the e-juice chamber, the wick in coupled to the second heating element;
a combined vapor chamber connected to the vapor cooling chamber, wherein a first valve in an open position allows fluid communication therebetween and in a closed position blocks fluid communication therebetween;
the combined vapor chamber connected to the coil chamber, wherein a second valve in an open position allows fluid communication therebetween and in a closed position blocks fluid communication therebetween;
a mouthpiece connected to the combined vapor chamber; and
a power source for providing electrical power to the first heating element and the second heating element.

6. A dual vaporizer according to claim 5, wherein relative opening of the first valve and the second valve are controllable to select a proportion of vapor entering the combined vapor chamber from the vapor cooling chamber and the coil chamber.

7. A method of vaporizing volatiles from Cannabis plant material comprising:
a. providing a dual vaporizer according to claim 6;
b. placing Cannabis plant material in the plant matter compartment;
c. filling the e-juice chamber with e-juice comprising at least one flavouring and at least one smoke-generating carrier;
d. heating the first heating element to vaporize volatiles in the Cannabis plant material;
e. heating the second heating element to vaporize the e-juice;
f. drawing the Cannabis plant material volatiles vapor through the second chamber to combine with the e-juice vapor generated therein to mask the odor and/or taste of the Cannabis plant material volatiles vapor prior to inhaling the vapors through the mouthpiece.
